(19) United States
(12) Patent Application Publication
(10) Pub. No.: US 2021/0401675 A1
(43) Pub. Date: Dec. 30, 2021

(54) Intimate Wipes

(71) Applicant: Carmen Agustina Cunningham Rivera, San Diego, CA (US)

(72) Inventor: Carmen Agustina Cunningham Rivera, San Diego, CA (US)

(21) Appl. No.: 17/022,618

(22) Filed: Sep. 16, 2020

Related U.S. Application Data
(60) Provisional application No. 63/045,249, filed on Jun. 29, 2020.

(51) Int. Cl. A61K 8/02; A41D 13/11; A47K 10/32; A01N 25/34; A01N 43/80

(52) U.S. Cl. CPC A61K 8/0208; A41D 13/1161; A47K 10/32; A47K 2010/3266; A01N 25/34; A01N 43/80; A41D 13/1192

(57) ABSTRACT

This present invention relates to an improved sanitary wipe usable in different application to clean the hands, the face, and other areas of the body, and to provide face mask functionality. The sanitary wipes are preferably made from pre-moistened biodegradable and flushable materials designed to not adversely affect the user's skin. The sanitary wipes can be stored in different types of packages for different applications, and can be dispensed from a roll and torn away individually along a plurality of spaced apart perforations. In an alternative embodiment, the sanitary wipes can also be individually pulled from a pack and applied to the skin as needed.

### CROSS-REFERENCE TO RELATED APPLICATION

The present application claims priority to, and the benefit of, U.S. Provisional Application No. 63/045,249, which was filed on Jun. 29, 2020 and is incorporated herein by reference in its entirety.

### BACKGROUND

The present invention relates generally to the field of wet or moist wipes. More specifically, the present invention relates to a novel, pre-packaged set of multipurpose sanitary wet wipes. The wet wipes are useful for cleaning the hands, face, and other areas of the body. Each individual wipe of the product is pre-moistened, and is made up of a biodegradable and flushable material. The wipes may be stored in, and dispensed from, different types of packages for different applications as per the preferences of the users, and based on the particular end use application. The present invention further comprises a portable package that enable users to apply the wet wipes in any situation that requires sanitization or cleansing on the go. Further, the individual wet wipe may also function as a face mask to protect the wearer against airborne pathogens, pollution, microbes, particulates, and other debris. In one embodiment, the package of wet wipes may be available in the form of a roll that is stored in a mechanism similar to a toilet paper roll holder or dispenser. The individual wet wipe is soft, fully clean and sanitized, and disinfects the user's skin without causing irritation. The package containers are also reusable, and may feature a sealed lid with a moveable closure to prevent the wipes from drying out prior to their use. Accordingly, the present specification makes specific reference thereto. However, it is to be appreciated that aspects of the present invention are also equally useable with other like applications, devices and methods of manufacture.

In the area of personal hygiene and cleanliness, several cleaning aids and devices dominate consumer choices because of their relative low cost and versatility. Specifically, consumers typically reach for toilet paper, facial tissues, and pre-moistened wipes to meet personal hygiene needs. These all-purpose products are designed for minimal effort by the average consumer in order to achieve the desired cleaning, and are relatively inexpensive to procure and use.

However, adequate cleaning of the skin is a personal hygiene issue that is not always easily resolved with a “dry” product. By way of background, dry tissue products are the most commonly used cleansing products post-defecation, post-urination and during menstruation. Dry tissue products are also commonly used to remove soils or stains, such as those caused by food, dirt and the like, from the skin. Dry tissue products are generally referred to as “toilet paper,” “toilet tissue”, “paper tissue” or “paper towels.” Unfortunately, because such products are dry (i.e., lack moisture or wetness), they are not particularly effective at removing certain stains, such as dried stains. Also, such dry products also tend to easily deteriorate if rubbed on a surface, for example, to remove a difficult spill or stain.

Wet products, such as wet wipes, provide certain advantages over commonly available dry tissue products, and are useful for many different applications. For example, wet wipes may be used when changing diapers of small children and infants, for household cleaning tasks, for cleaning hands, as a bath tissue, to clean a disabled, infirm, sick, aged or incontinent adult by a caregiver, and may be used in and for a whole host of other applications where it is advantageous to have a sanitary wipe or towel that has some moisture in it. Other wet wipes are used for the removal of makeup, sunscreen or other cosmetic or topical products that have been applied to the skin, and that require removal after a period of time. Unfortunately, wet wipes may not always be easily accessible, especially outside the home, and may be difficult to keep moist during transportation so that the wipes do not dry out prior to their use. Further, historically, wet wipes have not been flushable, meaning that they tend to clog the drain if flushed in, for example, a toilet or other sanitary sewer or septic system.

Furthermore, most bathrooms have standard toilet tissue paper stored on a toilet paper roll holder for easy dispensing. However, after going to the bathroom, people may desire more extensive cleaning methods for the body than just standard, dry toilet tissue paper. For example people may desire a roll of wet wipes in a toilet paper-style holder, usable after going to the bathroom to clean any area of the body with a moist towelette. Wet wipes clean, sanitize and disinfect the region after restroom use, and the added moisture enables the user to fully cleanse the area.

Additionally, during the ongoing global pandemic, face masks have become a necessity for individuals to protect themselves from airborne pathogens, microbes, pollution and other debris whenever they leave the safety of the home or other protected space. However, having to continuously carry a face mask for such applications is inconvenient for the user. Further, if the individual forgets to carry the facemask, he or she may find themselves without adequate protection, and/or be shamed for not wearing a mask in public.

One particular shortcoming of many commercially available personal hygiene cleaning products on the market is the containers and dispensers in which the products are sold or distributed in. For example, the typical toilet paper holder does not have a restricting mechanism to prevent the entire roll from being unraveled, and is incapable of dispensing, for example, wet wipes in a moist condition. Additionally, some such containers are bulky, and can be difficult and heavy to carry for prolonged periods of time (i.e., during extended travel).

Personal hygiene products currently on the market are also limited in their ability to provide quick and easy cleaning. Some products, such as toilet paper and facial tissue, are not pre-moistened with a hygienic or sanitizing cleaning solution. The average consumer may need a cleaning aid that is already premoistened upon dispensing. More specifically, when the dispensed personal hygiene product is already impregnated or infused with a mild cleaning or sanitizing solution, the coordination, strength, and time required to clean the body surface may be reduced by having such a product readily available.

Further, as previously stated, most pre-moistened sanitary wipe products on the market today are not flushable, and may also not be safe for use in septic based systems. More specifically, such products tend to cause problems by blocking or clogging pipes, or downstream at the sewage treatment facility where filters and other apparatus can become jammed or less effective over time due to the accumulation of such products.

Therefore, there exists a long felt need in the art for a biodegradable and disposable sanitary wet wipe usable in almost any situation to clean the hands, face, and other areas of the body or other surfaces. There is also a long felt need in the art for an improved sanitary wet wipe that is manufactured from pre-moistened, biodegradable, flushable materials which may be disposed of in the public sewage treatment system and that are septic system safe. Additionally, there is a long felt need in the art for an improved sanitary wipe that does not dry out before being used, that is it maintains its moist condition for longer periods of time within the container or dispenser. Moreover, there is a long felt need in the art for a sanitary wipe that also functions as a face mask to protect the wearer against airborne pathogens, microbes, pollution and other debris, and so that the user does not have to carry a separate facemask. Further, there is a long felt need in the art for a sanitary wipe that may be stored in and dispensed from a mechanism that is similar to a toilet paper roll holder so that the wipe may be torn away or separated individually as needed along perforations or die cut lines. Finally, there is a long felt need in the art for a sanitary wipe that is easily accessible and easy to transport and carry, and that enables users to apply the wet wipe in any situation that requires sanitization or disinfection.

The subject matter disclosed and claimed herein, in one embodiment thereof, comprises a multipurpose sanitary wet wipe product that is pre-moistened and comprised of a biodegradable and flushable material. The sanitary wipe of the present invention may be: (a) stacked in a tissue style package having a top surface, an enclosure on the top surface having a sealed and moveable lid that can be opened to create a dispensing opening for individual removal of the wet wipe; (b) available in a roll, and stored in a mechanism similar to a toilet paper roll holder; and (c) available as a protective face mask to protect the wearer against airborne pathogens, microbes, pollution and other harmful particulates.

The multipurpose sanitary wet wipe product of the present invention offers a way for men and women of all ages to maintain proper sanitary conditions of the hands, face, and other areas of the body, at any necessary time and in any situation. The sanitary wipe is easily accessible, easy to store and carry, and can be applied multiple times a day to the skin to help eliminate odors, soothe skin, and maintain personal care and hygiene. The sanitary wipes may also be used as a temporary bathing solution for those without immediate access to a shower or running water, such as with military activity, festival goers, camping, outdoor events, and more or just in those situations where an individual needs to feel clean.

In this manner, the sanitary wipe product of the present invention accomplishes all of the forgoing objectives, and provides a relatively safe, easy, convenient and cost-effective solution. The sanitary wipe product of the present invention is also user friendly inasmuch as it is relatively inexpensive, disposable, flushable, and does not require the user to put much effort to help eliminate odors, soothe skin, and maintain personal hygiene and care.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

As used herein, the term “wet wipe” refers to a fibrous, paper, cloth sheet, woven or non-woven material that is pre-moistened with a mild aqueous anti-bacterial or anti-microbial solution, and that is made of biodegradable and flushable materials. The amount of solution contained within each wet wipe can vary depending upon the type of material being used to provide the wet wipe, the type of container being used to store the wet wipes until needed, and the desired end use of the wet wipe. The solution may also include one or more of a fragrance, a cleaning agent, a sanitizer, an anti-bacterial agent, a preservative, and/or an emollient.

The subject matter disclosed and claimed herein, in one embodiment thereof, relates to a pre-moistened package of sanitary wet wipes that are stacked interfolded or integrally interconnected in a tissue-style container having a top surface, an enclosure on the top surface having a lid that can be opened to create an opening for removal of an individual sanitary wet wipe. The individual wet wipe is pre-moistened with a mild aqueous anti-bacterial or anti-microbial solution. Further, the container is reusable and features a lid that can seal the container to prevent the wipes from drying out prior to their use. The tissue-style container is portable, lightweight and easy to carry, and enables users to use and apply the sanitary wet wipes in any situation that requires sanitization, for example, while away from home.

In a further embodiment of the present invention, a supply roll of pre-moistened wet wipes is provided, wherein the roll of wet wipes is further comprised of a plurality of perforated or tear lines for separating the individual sanitary wipes as needed. The roll may be housed or suspended in a toilet paper-style wet wipes holder. More specifically, the roll of the wet wipes is installed in the toilet paper-style holder so that the wipes are dispensed from the roll and torn away individually at regularly spaced perforations. The sanitary wet wipes are infused with a solution that enable the same to be used to clean/sanitize a region after restroom use, and the added moisture enables the user to fully cleanse the area.

In a further embodiment of the present invention, a pre-moistened wet wipe face mask is disclosed that is specifically designed to protect the wearer against airborne pathogens, microbes, pollution and other debris and harmful particulates. The facemask is created from an individual wet wipe that is further comprised of both left ear and right ear loops. The ear loops are made up from narrow strips of a suitable material, such as a combination of textured polyester and lycra spandex, elastic, rubber, string or any other flexible material. The wet wipe portion of the facemask application may have pleats in the horizontal axis (i.e., across the face, as opposed to up and down), and be sized and shaped to fit over the mouth and nostrils of the wearer. Each ear loop is sewn or alternatively attached to the wet wipe portion by, for example, heat bonding, an adhesive, etc.

In yet another embodiment of the present invention, a method of using a wet wipe of the present invention is disclosed to clean the hands, face, and other areas of the body. The steps including acquiring a wet wipe product from the stacked wipes present in a container, opening the sealed lid thereby creating a dispensing opening, taking an individual wet wipe out of the opening, and cleaning the desired portion of the body and skin.

In a still further embodiment of the present invention, a combination sanitary wipe and dispensing container is described and includes a supply of biodegradable sanitary wipes with the supply being equally divided into a series of individual wipes, wherein each of the individual wipes are separable from the supply by a line of perforation having a series of cuts and ties. The combination includes a container sized and configured to receive the supply of sanitary wipes, wherein the container has an opening therein to dispense the individual wipes from the supply. Additionally, a solution is infused into the supply of sanitary wipes, the solution comprising one of more of the following: a water, an emollient, a perfume, a skin care agent, a bactericide, a fungicide, and a methylisothiazolinone.

In a yet still further embodiment of the present invention, a disposable face mask product is described, and includes a supply of non-woven material formed from cellulosic fibers. The supply of material is equally divided into a plurality of individual sheets of material by lines of perforation. Each of the sheets of material having left and right ear loops for securing each of the individual sheets to a face of a user. A solution is infused into the supply of individual sheets, with the solution providing protection from airborne microbes, pathogens, pollution and other harmful particulates. This particular embodiment also includes a dispenser for dispensing the individual sheets in an efficient and easy to use manner.

The sanitary wipes of the present invention are particularly advantageous because the same can be used by men and women of all ages in any situation, such as outside of the home or workplace, on the go, etc., to help eliminate odors, soothe skin, and to clean the hands, face, and other areas of the body.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1a illustrates a perspective view of one potential embodiment of a pre-packaged set of multipurpose sanitary wet wipes of the present invention in accordance with the disclosed architecture, wherein the container is in the closed position;

FIG. 1b illustrates a perspective view of one potential embodiment of a pre-packaged set of multipurpose sanitary wet wipes of the present invention in accordance with the disclosed architecture, wherein the container is in the open position;

FIG. 2a illustrates a perspective view of one potential of a sanitary wet wipe and dispenser combination of the present invention in accordance with the disclosed architecture;

FIG. 2b illustrates a perspective view of the sanitary wet wipe and dispenser combination of FIG. 2a, wherein a user is about to tear-off an individual wet wipe along a perforated line;

FIG. 3a illustrates a perspective view of different potential embodiments of individual sanitary wet wipes of the present invention in accordance with the disclosed architecture;

FIG. 3b illustrates a perspective view of different potential embodiments of individual sanitary wet wipes of the present invention in accordance with the disclosed architecture;

FIG. 4 illustrates a perspective view of one potential embodiment of a sanitary wet wipe disposable facemask of the present invention in accordance with the disclosed architecture, and being held by a user;

FIG. 5 illustrates a perspective side view of one potential embodiment of a sanitary wet wipe disposable facemask of the present invention in accordance with the disclosed architecture, and being worn by a user;

FIG. 6 shows a side elevational view of one potential embodiment of the separate ear loops of the sanitary wet wipe disposable facemask of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention or do not limit the scope of the invention. Additionally, an illustrated embodiment need not have all the aspects or advantages shown. Thus, in other embodiments, any of the features described herein from different embodiments may be combined.

As noted above, there exists a long felt need in the art for an improved and disposable sanitary wet wipe for cleaning the hands, face, other areas of the body or a surface. There is also a long felt need in the art for an improved sanitary wet wipe comprised of pre-moistened biodegradable, and flushable material, and that does not dry out before being used. Additionally, there is a long felt need in the art for an improved sanitary wipe that functions as a face mask to protect the wearer against airborne pathogens, pollution and other debris so that user does not have to carry a separate facemask. Finally, there is a long felt need in the art for an improved sanitary wipe stored in a dispensing mechanism that is similar to a toilet paper roll holder, wherein individual wipes may be torn away along perforations.

The present invention, in one exemplary embodiment, is a pre-packaged set of multipurpose sanitary wet wipes. The wet wipes can be used to clean the hands, face, and other areas of the body. Each individual wipe is pre-moistened and infused with a unique cleaning solution, and is comprised of a biodegradable and flushable material. The sanitary wipes may be stored in, and dispensed from, different types of packages for different applications to suit user need and/or preference. The present invention also features a portable package that enables users to apply the wet wipes in any situation that requires sanitization or cleaning, while away from home. In a further embodiment, the individual wet wipe also functions as a face mask to protect the wearer against airborne pathogens, microbes, pollution and other debris and harmful particulates. In one version, the package is available in a roll form which is stored in a mechanism similar to a toilet paper roll holder for ease of dispensing. The individual wet wipe is soft, fully clean and sanitized, and disinfects the skin without causing irritation thereto. Moreover, the package containers are reusable, and may feature a sealed lid to prevent the wipes from prematurely drying out.

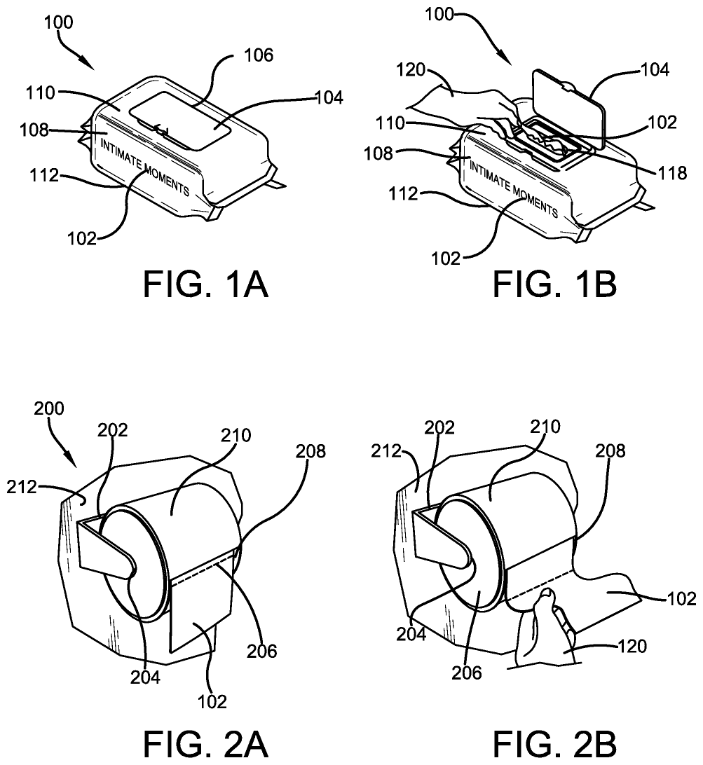

Referring initially to the drawings, FIG. 1a illustrates a perspective view of one potential embodiment of a pre-packaged set of multipurpose sanitary wet wipes 102 of the present invention in a closed container 100. As explained more fully below, container 100 is useful for housing and dispensing the wipes 102 until needed, and FIG. 1b illustrates a perspective view of the container 100 in the open position and ready to dispense the individual sanitary wipes 102. As shown in FIG. 1, the container 100 is of a generally cuboidal shape that includes a body generally made up of durable and flexible plastic-based packaging material, such as poly bags made from polyester or polypropylene material and the like. The packaging material for the container 100 forms a complete package for the pre-moistened wet wipes 102. More specifically, the container 100 has a top surface 110, elongated side portions 108 (which may be further comprised of a logo and/or name 114 of the product, such as “Intimate Moments”), a bottom surface 112 on which the container rests on a surface, a sealed lid 104 which is pivotally hinged on an edge 106 to open and close the container 100 as needed to dispense the individual sanitary wipes 102 as needed. The sealed lid 104 is preferably airtight, but is at least sufficiently sealed to prevent spoilage and drying out of the sanitary wipes 102 within the container 100.

The sanitary wet wipes 102 are preferably stacked in an interfolded, z-fold or c-fold configuration, or integrally interconnected, within the container 100, so as to allow for efficient and easy removal of a single sanitary wipe 102. As best shown in FIG. 1b, once the sealed lid 104 is opened along the hinged edge 106, a dispensing opening 118 is created through which a user 120 can take out an individual sanitary wipe 102 from the stacked wipes housed within the container 100. In a preferred embodiment, the sealed lid 104 may have a locking tab 105 to lock the lid 104 in the closed position until needed. Notwithstanding, other suitable closure mechanisms may also be used including, without limitation, an adhesive, hook and loop fasteners, buttons, clasps, snaps, etc.

Additionally, the lid 104 may be permanently or removably attached to the container 100. For example, in one embodiment the lid 104 may be entirely removable to allow for the container 100 to be refilled with sanitary wipes 102, thereby making the container reusable and extending its useful life and reducing the waste stream. There are many alternative means for attaching the sealed lid 104 to the enclosure 110 of the container. For example, the lid 104 may be manufactured with one or more ridges that can be snap-fitted into corresponding receiving grooves in the enclosure 110. Alternatively, the lid 104 may be designed so that it can be friction-fitted to the enclosure 110.

The container 100 itself may be of any suitable shape and size including, but not limited to, rectangular, cubic, pyramidal, cylindrical, spherical, or any other shape that allows a supply, such as a stack or roll of wet wipes 102 to be stored therein. Moreover, the container 100 is portable, relatively lightweight, and can be easily carried while traveling or away from a convenient bathing location. Although the container 100 is generally made of plastic material, such as a polyethylene, in other embodiments, the container 100 may be made of any suitable material, such as fabric products with a waterproof lining, waxed or poly-coated paperboard or the like to maintain the moisture of the sanitary wipes 102 stored therein. The container 100 may also be of any color and design as per the preferences of the user and/or the manufacturer.

As noted above, the sanitary wipes 102 may be arranged in the container 100 as a plurality of individual sheets arranged in a stacked configuration to provide a stack of wipes which may or may not be individually folded. The sanitary wipes 102 may be individual wipes which are folded in a c-fold, z-fold, quarter fold or other zigzag fold, or that are interfolded or non-interfolded as is known to those skilled in the art. The plurality of sanitary wipes 102 may also be stacked one on top of each other in a non-interfolded configuration for easy “reach-in” dispensing. More specifically, in a non-interfolded wipe configuration, each of the sanitary wipes 102 is folded onto itself with no portion of another adjacent wipe being positioned between or underneath any portion of the folds of the adjacent wipe(s). In this manner, the stack of folded sanitary wipes 102 remains wet or moist upon removal from the container 100.

The container 100 can include any suitable number of individual wipes 102 depending upon the desired packaging and intended end use. For example, the container 100 can be configured to include a stack of sanitary wipes 102, which range in number from about 10 to about 300 individual wipes, and more preferably from about 20 to about 80 wipes. The size and shape of the container 100 is dependent upon the size and shape of the stack of sanitary wipes 102 to be provided therein, and vice versa.

FIG. 2a illustrates a perspective view of one potential of a sanitary wet wipe and dispenser combination of the present invention in accordance with the disclosed architecture, and FIG. 2b illustrates a perspective view of the sanitary wet wipe and dispenser combination of FIG. 2a, wherein a user is about to tear-off an individual wet wipe along a perforated line. More specifically, a continuous supply or roll of sanitary wipes 102 are stored in a dispensing mechanism 200 similar to a toilet paper roll holder. As best shown in FIG. 2a, the toilet paper roll holder type dispenser 200 may have a back mounting plate 202, a rounded or curved body portion 210, a gap or opening 208 in the curved part 210 from which to dispense the sanitary wipes 102.

The dispenser 200 may be mounted against a wall 212 by means of the back mounting plate 202, which can be attached to the curved portion 210 using, for example, a screw and a nut 204. The back-mounting plate 202 may be removably attached to the curved portion 210. Each sanitary wipe 102 has perforations 206 where the individual wipe 102 is torn away from the rest of the supply wall. The curved portion 210 may be transparent for viewing the number of sanitary wipes 102 that remain in the dispenser 200. The curved portion 210 also forms a cover for the sanitary wipes 102. Therefore, it is preferred that all materials that are in contact with or associated with the sanitary wipes 102 be comprised of materials that discourage, or do not support, bacterial growth.

The roll of sanitary wipes 102 that are useful with the dispenser 200 may come in various sizes and lengths. For example, the length of the wipes comprising the roll may vary from a few linear inches to more than a thousand linear inches of sanitary wipes 102. The individual sanitary wipes 102 that comprise the roll are separated by perforations 206 that enable individual wipes 102 to be easily torn and separated from the roll. Perforations 206 refer to the amount of partial cutting and the distance between the partial cuts in the roll that enables each of the wipes 102 in the roll to be separated. One example of a roll that is particularly useful for applications in the home is one that has a diameter of approximately five inches, and that is comprised of perforation lines 206 providing somewhere between 6% and 30% hold between the individual wipes 102 (meaning 6-30% of the strength the roll would normally exhibit if there were no perforations). To achieve this degree of hold, the line of perforations 206 should have between two and ten partial cuts per linear inch, and the individual ties (which refers to the portions of the wipe 102 between the partial cuts but still in the perforation line) preferably have a length that ranges from 0.020″ to 0.040.″

In a preferred embodiment of the present invention, a slotted moisture protective barrier may extend across the opening 208 of the wet wipe dispenser 200, as best shown in FIG. 2a. The protective barrier across the opening 208 helps to ensure that excessive air does not enter the chamber or interior space of the dispenser and cause the sanitary wet wipes 102 to dry out prematurely. While many materials are suitable, preferably the barrier is of a polymeric material, that is both flexible and transparent.

FIG. 2b illustrates a perspective view of the sanitary wet wipe and dispenser combination of FIG. 2a, wherein a user 120 is about to tear-off an individual wet wipe 102 along a perforated line 206 as the roll extends through the opening 208 of the dispenser 200. The roll of wipes 102 may unwind in either a clockwise or counterclockwise manner to suit the user's 120 need or preference. The sanitary wipe 102 has sufficient wet strength for its intended use, but is comprised of material that will disintegrate when flushed and subject to the water treatment system and other septic bacterial action.

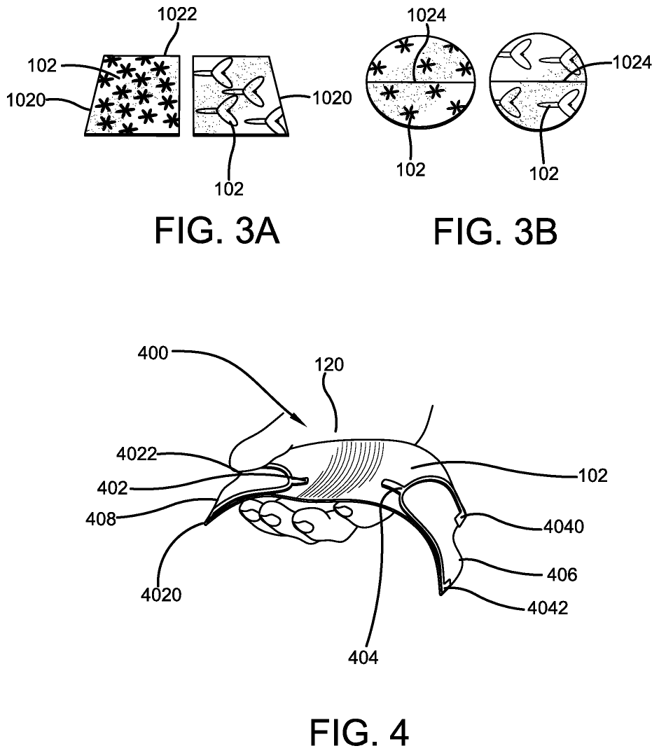

FIGS. 3a and 3b illustrate differing perspective views of different potential embodiments of individual sanitary wet wipes 102 of the present invention in accordance with the disclosed architecture. More specifically, FIG. 3a illustrates sanitary wet wipes 102 that are generally rectangular or square in shape, and that may have any desired unfolded width and/or length. For example, the wet wipe 102 may have an unfolded length 1020 of from about 3 inches to about 10 inches, and an unfolded width 1022 of from about 2 inches to about 6 inches.

As best shown in FIG. 3b, each wet wipe 102 may also be generally circular or oval in shape, and may have any desired unfolded diameter 1024. For example, the circular wet wipe 102 may have an unfolded diameter 1024 of from about 2 inches to about 6 inches. In a preferred embodiment, the individual sanitary wet wipe 102 may have an aspect ratio of length to width of between 1 and 6. Alternatively, the sanitary wet wipes 102 may include a continuous supply of material (e.g., a roll) which has perforations 206 between each individual wipe 102, and that which may be used in conjunction with dispenser 200, as described above. Nonetheless, in any of the forgoing embodiments, the sanitary wet wipes 102 may be used on both sides with equal efficiency and usefulness.

The appearance, design, color of each side of the wet wipe 102 may be the same or different as per the wants and needs of the user. Additionally, as previously stated, each individual wet wipe 102 may be made from a variety of eco-friendly, biodegradable and flushable materials. For example, the sanitary wipe 102 is preferably comprised of a non-woven material having fibrous materials that provide an improved appearance and feel to the wet wipe 102. Natural fibers, such as cellulosic fibers, natural plant fibers, those fibers marketed under the brand VESTERA®, etc., may also be used in the construction of the sanitary wipes 102 of the present invention. Other post-consumer waste products may also be suitable provided that the same are sterilized and treated before use in the present invention. Thermoplastic polymeric fibers such as polyolefins may also be used in the present invention.

As previously noted, each sanitary wet wipe 102 may be moistened with water or other liquids and solutions, such as isopropyl alcohol, depending on the intended application. The sanitary wet wipes 102 may also be treated with softeners, lotions, or perfumes to adjust the tactile and olfactory properties of the wipe 102. Preservatives, such as methylisothiazolinone, may also be used to prevent bacterial or fungal growth on the wipe 102 prior to its use. The sanitary wipes 102 may further comprise texturing or patterning on the surface, as best shown in FIGS. 3a and 3b, to allow for additional cleaning and/or to improve the aesthetic appearance of the sanitary wipe 102.

In other embodiment, the wipe 102 is impregnated with a wetting composition containing ingredients that relate to the intended use of the wipe 102. While a major component of the wetting composition by weight is typically water, other components may include, without limitation, a cleansing agent, a skin care agent, a bactericide, an alcohol, a fungicide, an emollients, a perfume, a preservative etc. When appropriate, the solution may be manufactured in an alcohol-free version.

FIG. 4 illustrates a perspective view of one potential embodiment of a sanitary wet wipe disposable facemask 400 of the present invention in accordance with the disclosed architecture, and being held by a user. The pre-moistened wet wipe that functions as a face mask 400 may be used to protect the user against airborne pathogens, microbes, pollutions, harmful particulates and other debris. More specifically, the sanitary wipe facemask 400 comprises an individual pre-moistened wet wipe 102, an attached left ear loop 404, and an attached right ear loop 402. The respective left and right ear loops 402, 404 are preferably comprised of relatively narrow strips of a suitable material, such as a combination of textured polyester, lycra, spandex, elastic, rubber band, string, etc. The wet wipe 102 used for a facemask application may be further comprised of pleats along the horizontal axis (i.e., across the face, as opposed to up and down or vertically), and is dimensioned to fit over the mouth and nostril area of the wearer. The left ear loop 404 is sewn, or alternatively attached through a mechanism such as heat bonding, an adhesive or the like, to one corner 4040 and to an opposite corner 4042 on a first outer edge 406. Similarly, the right ear loop 402 is sewn or alternatively attached to one corner 4020 and to an opposite corner 4022 on a second outer edge 408. The ear loops 402, 404 extend outwardly from the respective edges to be engaged around the wearer's ears, and are generally symmetrical relative to the wet wipe portion 102 of the face mask 400.

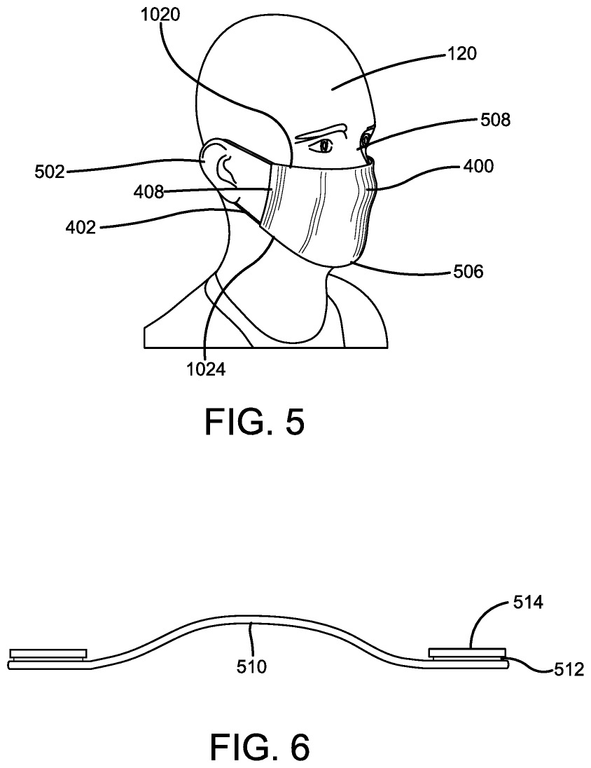

FIG. 5 illustrates a perspective side view of one potential embodiment of a sanitary wet wipe disposable facemask 400 of the present invention in accordance with the disclosed architecture, and being worn by a user 120. Similar to the mask shown in FIG. 4, the face mask 400 depicted in FIG. 5 is comprised of a generally rectangularly shaped wet wipe 102, a right ear loop 402 attached to the second outer edge side 408 and designed to fit over the right ear 502 of the wearer 120, wherein the material used to comprise both the right ear loop 402 and the wipe portion 102 allows for expansion. Similarly, the left ear loop 404 is attached to the first outer edge 406 and is designed to fit over the left ear (not shown) of the wearer 120. In this manner, the lower end of the wipe 102 can expand down over the chin 506 and is designed to accommodate the face of the wearer 120, and the upper portion 1020 of the wipe 102 may extend up over the bridge of the nose 508 of the wearer 120. The mask 400 may be for a single use application, or it may be used multiple times, thereby providing a cost effective solution to protecting a user from airborne pathogens, germs, microbes, particulates, and the like.

In an alternate embodiment, as shown in FIG. 6, the ear loops 510, which make up the left and right ear loops 402 and 404 of the earlier figures, may be provided separately with adhesive strips 512 covered by a release liner 514 so that the user can create a face mask 400 from the separate ear loops 510 and sanitary wet wipe 102 by removing the release liner 514 covering the adhesive, and attaching one end of the ear loops to each of the top and bottom corners of the sanitary wipe 102, and then repeating the attachment for the other side of the sanitary wipe 102, thereby creating the left and right ear loops 402 and 404.

The wet wipes 102 will be available in different sizes and colors to accommodate different needs and preferences. Exact size, measurement, construction and design specifications of the unique wipe 102 of the present invention may vary upon manufacturing or the particular material that is used, and different sizes may be provided such as to accommodate children, adults, men and women, pre-teens and teenagers. Further, the surface of the sanitary wipe 102 may be essentially flat, or it may optionally contain raised and/or lowered portions. The sanitary wipe 102 may also have logos, indicia, trademarks, geometric patterns, customizable colors, fonts, embroidery, textures and prints and/or images embossed therein or printed on their surface.

The strength properties of the pre-moistened wet wipes of the present invention make the wipe stronger in the direction typically associated with wiping (i.e. the length direction of the individual wet wipe product). This avoids breakdown of the sanitary wipe 102 during its use. The sanitary wipe may also be relatively weak in the width direction, which is normally under less stress during wiping, thereby making it possible for the sanitary wipe 102 to break up and disperse in the sewage after use (i.e., so as to not clog pipes, toilets and the like).

The container to hold the wet wipes of the present invention may include, but are not limited to, flow wrap pouches, individual sachets, chained sachets comprising a tear or perforated line between each sachet, and other forms known in the art as suitable for storing nonwoven articles. Additionally, the container may also be manufactured to facilitate removal of individual wet wipes.

With respect to the above description, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Certain terms are used throughout the description and the claims to refer to particular features or components. As one skilled in the art will appreciate, different persons may refer to the same feature or component by different names. This document does not intend to distinguish between components or features that differ in name, but not structure or function. As used herein “sanitary wipes”, “intimate wipes”, wet wipes”, and “multipurpose sanitary wet wipes product” are interchangeable and refer to the multipurpose sanitary pre-moistened wet wipes 102 product of the present invention.

Notwithstanding the forgoing, the sanitary wet wipe product 102 of the present invention can be of any suitable size and configuration as is known in the art without affecting the overall concept of the invention, provided that it accomplishes the above stated objectives. One of ordinary skill in the art will appreciate that the size, configuration and material of the wet wipes 102 as shown in the FIGS. are for illustrative purposes only, and that many other sizes of the wet wipes 102 are well within the scope of the present disclosure. Although the dimensions of the wet wipes 102 are important design parameters for user convenience, the wet wipes 102 may be of any size that ensures optimal performance during use and/or that suits pet owners need and/or preference.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A sanitary wipe product comprising;
a supply of material divided into a plurality of equally sized wipes via a plurality of perforation lines, wherein the supply of material is biodegradable;
a solution including a water, an emollient, and a perfume, wherein each of the plurality of equally sized wipes is infused in with the solution; and
the supply of material is enclosed in a container.

2. The sanitary wipe product of claim 1, wherein each of the plurality of perforation lines has a plurality of cuts and a plurality of ties, wherein the plurality of cuts are between 2 and 10 cuts per inch, and each of the plurality of ties have a length ranging from 0.020″ to 0.040″.

3. The sanitary wipe product of claim 1, wherein each of the plurality of equally sized wipes further comprise a pair of ear loops to form a face mask.

4. The sanitary wipe product of claim 3, wherein each of the pair of ear loops comprise an adhesive covered by a release liner for securement to each of the plurality of equally sized wipes.

5. The sanitary wipe product of claim 1, wherein the solution further comprises a preservative.

6. The sanitary wipe product of claim 1, wherein the preservative is a methylisothiazolinone.

7. The sanitary wipe product of claim 1, wherein at least one of the plurality of equally sized wipes is comprised of at least one of a texture, a pattern, a graphic, a logo, an indicia, a trademark, a geometric pattern, a color, a font, and an embroidery.

8. The sanitary wipe product of claim 1, wherein not all of the plurality of equally sized wipes are equally sized.

9. The sanitary wipe product of claim 1, wherein the solution is further comprised with at least one of a skin care agent, a bactericide, and a fungicide.

10. A combination sanitary wipe and dispensing container comprising:
a supply of biodegradable sanitary wipes being equally divided into a series of individual wipes, wherein each of the series of individual wipes is separable from the supply of biodegradable sanitary wipes by a line of perforation having a plurality of cuts and a plurality of ties;
a container sized and configured to receive the supply of biodegradable sanitary wipes, wherein the container comprises an opening to dispense the series of individual wipes; and
a solution infused into the series of individual wipes.

11. The combination sanitary wipe and dispensing container of claim 10, wherein a separate supply of ear loops is provided with the combination to form a face mask.

12. The combination sanitary wipe and dispensing container of claim 11, wherein each of the separate supply of ear loops is comprised of an adhesive and a release liner.

13. The combination sanitary wipe and dispensing container of claim 10, wherein the solution is comprised of more than one of a water, an emollient, a perfume, a skin care agent, a bactericide, a fungicide, and a preservative.

14. The combination sanitary wipe and dispensing container of claim 13, wherein the preservative is a methylisothiazolinone.

15. The combination sanitary wipe and dispensing container of claim 10, wherein the series of individual wipes comprise more than one of a texture, a pattern, a graphic, a logo, an indicia, a trademark, a geometric pattern, a color, a font, and an embroidery.

16. The combination sanitary wipe and dispensing container of claim 10, wherein the series of individual wipes is comprised of natural plant fibers and is a non-woven product.

17. The combination sanitary wipe and dispensing container of claim 10, wherein each of the lines of perforation have between 2 and 10 of the plurality of cuts per inch, and further wherein a length of each of the plurality of ties range between 0.020″ to 0.040″.

18. A disposable face mask product comprising:
a supply of non-woven material formed from a plurality of cellulosic fibers, wherein the supply of non-woven material is equally divided into a plurality of individual sheets by a plurality of perforated lines;
each of the plurality of individual sheets comprising a left ear loop and a right ear loop for securing each of the plurality of individual sheets to a face of a user;
a solution infused into the a plurality of individual sheets; and
a dispenser for dispensing the plurality of individual sheets.

19. The disposable face mask product of claim 18, wherein each of the plurality of perforated lines have a plurality of cuts ranging in number from 2 to 10 per inch and a plurality of ties, and further wherein a length of each of the plurality of cuts ranges from between 0.020″ to 0.040″.

20. The disposable face mask product of claim 18, wherein the solution is comprised of at least two of a water, an emollient, a perfume, a skin care agent, a bactericide, a fungicide, and a methylisothiazolinone.
